(19) United States
(12) Patent Application Publication
(10) Pub. No.: US 2020/0263128 A1
(43) Pub. Date: Aug. 20, 2020

(54) In Vitro Fertilization Media with Antiviral Medicine

(71) Applicants: Michael C. Doody, Knoxville, TN (US); Sean C. Doody, Birmingham, AL (US); Lindsay C. Doody, Birmingham, AL (US)

(72) Inventors: Michael C. Doody, Knoxville, TN (US); Sean C. Doody, Birmingham, AL (US); Lindsay C. Doody, Birmingham, AL (US)

(21) Appl. No.: 16/793,600

(22) Filed: Feb. 18, 2020

Related U.S. Application Data
(60) Provisional application No. 62/806,429, filed on Feb. 15, 2019.

(51) Int. Cl. C12N 5/073; A61B 17/435; C12N 5/00

(52) U.S. Cl. CPC C12N 5/0604; C12N 2500/30; C12N 5/0018; A61B 17/435

(57) ABSTRACT

Assisted Reproductive Technologies (ART) are well recognized technologies to allow couples or individuals with a desire for pregnancy to achieve their goals. ART is a group of technologies which may include but is not limited to in vitro fertilization; donor insemination; embryo, sperm or oocyte cryopreservation; embryo, sperm or oocyte thawing; embryo, sperm or oocyte transfer into the uterus of a recipient; isolation, preparation and transportation of sperm cells, eggs and embryos for later use; intracytoplasmic insemination; genetic studies and surrogacy. This invention is directed toward an assisted reproductive technology media incorporating one or more antiviral compounds or medicines, and more specifically relates to such media incorporating one or more antiviral compounds or medicines directed to the HHV-6A virus, the HHV-6B virus or other viruses which are members of the Human Herpesvirus group.

### CROSS-REFERENCE TO RELATED APPLICATIONS

This application claims priority to and claims the benefit of the filing date of U.S. Provisional Patent Application Ser. No. 62/806,429, filed Feb. 15, 2019 and entitled “IN VITRO FERTILIZATION MEDIA WITH ANTIVIRAL MEDICINE.” The entire content of U.S. Provisional Patent Application Ser. No. 62/806,429 is incorporated herein by reference.

### FIELD OF INVENTION

This invention is directed toward an assisted reproductive technology media incorporating one or more antiviral compounds or medicines, and more specifically relates to such media incorporating one or more antiviral compounds or medicines directed to the HHV-6A virus, the HHV-6B virus or other viruses which are members of the Human Herpesvirus group.

### BACKGROUND

Assisted Reproductive Technologies (ART) are well recognized technologies to allow couples or individuals with a desire for pregnancy to achieve their goals. ART is a group of technologies which may include but is not limited to in vitro fertilization; donor insemination; embryo, sperm or oocyte cryopreservation; embryo, sperm or oocyte thawing; embryo, sperm or oocyte transfer into the uterus of a recipient; isolation, preparation and transportation of sperm cells, eggs and embryos for later use; intracytoplasmic insemination; genetic studies and surrogacy.

A need is felt for an assisted reproductive technology media incorporating one or more antiviral compounds or medicines, and more specifically for media incorporating one or more antiviral compounds or medicines directed to the HHV-6A virus, the HHV-6B virus or other viruses which are members of the Human Herpesvirus group.

### SUMMARY

It has been discovered that the Human Herpesvirus 6A (referred to here as HHV-6A virus) and the Human Herpesvirus 6B (referred to here as HHV-6B virus) or other viruses which are members of the Human Herpesvirus group, if present in reproductive cells or media at the time of ART efforts to produce pregnancy, can cause a reduction in the likelihood of conception, or may increase the likelihood that the genetic material of HHV-6A or HHV-6B would adversely be integrated within the DNA of the embryo. Accordingly, it is within the scope of this invention to add one or more antiviral compounds or medicines to in vitro fertilization or other ART media that is specifically directed to inactivating or reducing the activity of the HHV-6A virus, the HHV-6B virus or other viruses which are members of the Human Herpesvirus group.

During the in vitro fertilization or other ART processes, the HHV-6A virus, the HHV-6B virus or other viruses which are members of the Human Herpesvirus group can potentially reduce the viability or function of oocytes, sperm cells and embryos.

Thus, in some embodiments of the present general inventive concept, an in vitro fertilization media with antiviral protection includes in vitro fertilization media incorporating at least one antiviral compound or medicine which targets the HHV-6A virus, the HHV-6B virus or other viruses which are members of the Human Herpesvirus group.

In some embodiments, said antiviral compound or medicine includes a compound selected from the list consisting of acyclovir, valciclovir, famciclovir, foscarnet, valganciclovir, penciclovir, and cidofovir.

In some embodiments, said antiviral compound or medicine includes a guanosine derivative.

In some embodiments, said antiviral compound or medicine includes a guanosine analogue.

In some embodiments, said antiviral compound or medicine includes a pyrophosphate analogue DNA polymerase inhibitor.

In some embodiments, said in vitro fertilization media includes media for the isolation of oocytes, ovum and other cells of ovarian origin during the egg retrieval process.

In some embodiments, said in vitro fertilization media includes media for the fertilization of ovum.

In some embodiments, said media for the fertilization of ovum includes media adapted to fertilization of ovum by sperm-egg interaction.

In some embodiments, said media for the fertilization of ovum includes media adapted to fertilization of ovum intracytoplasmic sperm injection.

In some embodiments, said in vitro fertilization media includes media for the preparation and isolation of sperm cells used in the in vitro fertilization process.

In some embodiments, said in vitro fertilization media includes media to sustain the viability of embryos of all stages and blastocysts from the time of fertilization until replacement in the uterus, embryo biopsy, embryo cryopreservation or any other ultimate disposition of the embryo.

In some embodiments, said in vitro fertilization media includes media to sustain the viability of other cells which may be grown together in co-culture of human oocytes and embryos of all stages of development.

In some embodiments, said in vitro fertilization media includes media used in the cryopreservation and thawing of human sperm cells, oocytes, ovum, embryos and blastocysts at all stages of development.

In some embodiments, said in vitro fertilization media includes media used in the maintenance and transportation of biopsied embryo cells for further analysis or use.

In some embodiments, said in vitro fertilization media includes media for the pre-conditioning of laboratory containers or catheters to be later used in the in vitro fertilization process.

In some embodiments, said in vitro fertilization media includes media used for the transfer of sperm cells, oocytes or embryos into the uterus of a recipient at the time of an ART procedure such as intrauterine insemination or embryo transfer.

From the foregoing, it will be recognized that an in vitro fertilization media has been provided that includes selected amounts of one or more antiviral compounds or medicines for targeting the HHV-6A virus, the HHV-6B virus or other viruses which are members of the Human Herpesvirus group. These viruses can be expected based upon current evidence to reduce the fertility of embryos, ovum, and/or sperm cells with which they contact. Thus, the in vitro fertilization media containing the antiviral compound or medication will be more likely to result in pregnancy.

### FIGURES

The above-mentioned and additional features of the invention will become more clearly understood from the following detailed description of the invention read together with the drawings in which:

FIG. 1 is a flow chart of one example embodiment of an in vitro fertilization process during which media according to one example embodiment of the present general inventive concept may be used.

### DETAILED DESCRIPTION

It has been discovered that the Human Herpesvirus 6A (referred to here as HHV-6A virus) and the Human Herpesvirus 6B (referred to here as HHV-6B virus) or other viruses which are members of the Human Herpesvirus group, if present in reproductive cells or media at the time of ART efforts to produce pregnancy, can cause a reduction in the likelihood of conception, or may increase the likelihood that the genetic material of HHV-6A or HHV-6B would adversely be integrated within the DNA of the embryo. Accordingly, it is within the scope of this invention to add one or more antiviral compounds or medicines to in vitro fertilization or other ART media that is specifically directed to inactivating or reducing the activity of the HHV-6A virus, the HHV-6B virus or other viruses which are members of the Human Herpesvirus group.

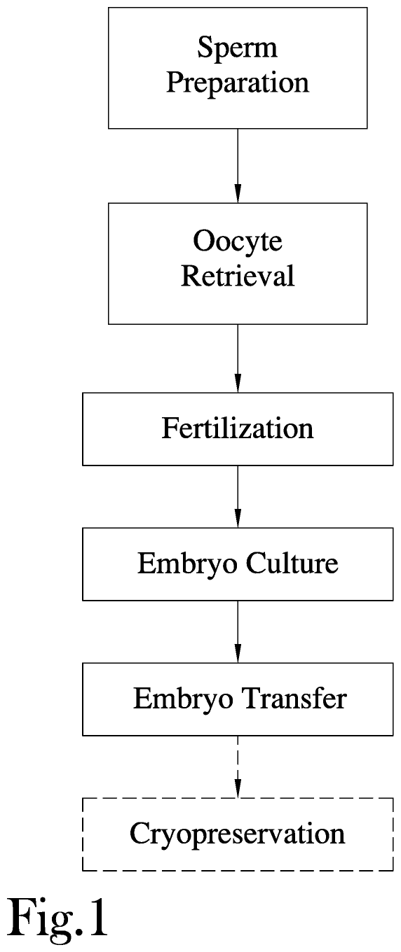

During the in vitro fertilization or other ART processes, the HHV-6A virus, the HHV-6B virus or other viruses which are members of the Human Herpesvirus group can potentially reduce the viability or function of oocytes, sperm cells and embryos. In accordance of various features of the present invention, one or more antiviral compounds or medicines can be used in one or more of the following media, used in various steps of the ART process illustrated generally in FIG. 1, or in a similar ART process or procedure:

Media for the isolation of oocytes, and other cells of ovarian origin during the egg retrieval process.

Media for the fertilization of ovum, whether by sperm-egg interaction, intracytoplasmic sperm injection, or other technologies.

Media for the preparation and isolation of sperm cells used in the in vitro fertilization process.

Media to sustain the viability of embryos of all stages and blastocysts from the time of fertilization until replacement in the uterus, embryo biopsy, embryo cryopreservation or any other ultimate disposition of the embryo.

Media to sustain the viability of other cells which may be grown together in co-culture of human oocytes and embryos of all stages of development.

Media used in the cryopreservation and thawing of human sperm cells, oocytes, embryos and blastocysts at all stages of development.

Media used in the maintenance and transportation of biopsied embryo cells for further analysis or use.

Media for the pre-conditioning of laboratory containers or catheters to be later used in the in vitro fertilization process.

Media used for the transfer of sperm cells, oocytes or embryos into the uterus of a recipient at the time of an ART procedure such as intrauterine insemination or embryo transfer.

From the foregoing, it will be recognized that an in vitro fertilization media has been provided that includes selected amounts of one or more antiviral compounds or medicines for targeting the HHV-6A virus, the HHV-6B virus or other viruses which are members of the Human Herpesvirus group. These viruses can be expected based upon current evidence to reduce the fertility of embryos, ovum, and/or sperm cells with which they contact. Thus, the in vitro fertilization media containing the antiviral compound or medication will be more likely to result in pregnancy.

In accordance with the present general inventive concept, antiviral compounds or medications contained within in vitro fertilization media in various example embodiments include guanosine derivatives and/or analogues, including famciclovir, cidofovir, and acyclovir. In various example embodiments, the antiviral compounds or medications contained within in vitro fertilization media include pyrophosphate analogue DNA polymerase inhibitors, such as foscarnet. In various example embodiments, the antiviral compounds or medications contained within in vitro fertilization media include one or more compounds or medications selected from the list consisting of acyclovir, valciclovir, famciclovir, foscarnet, valganciclovir, penciclovir, and cidofovir. Those of skill in the art will recognize that other antiviral compounds or medications could also be used in this context, and that those other antiviral compounds or medications also fall within the scope of the present general inventive concept.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

1. An in vitro fertilization media with antiviral protection, comprising:
in vitro fertilization media incorporating at least one antiviral compound or medicine which targets the HHV-6A virus, the HHV-6B virus or other viruses which are members of the Human Herpesvirus group.

2. The in vitro fertilization media with antiviral protection of claim 1, wherein said antiviral compound or medicine includes a compound selected from the list consisting of acyclovir, valciclovir, famciclovir, foscarnet, valganciclovir, penciclovir, and cidofovir.

3. The in vitro fertilization media with antiviral protection of claim 1, wherein said antiviral compound or medicine includes a guanosine derivative.

4. The in vitro fertilization media with antiviral protection of claim 1, wherein said antiviral compound or medicine includes a guanosine analogue.

5. The in vitro fertilization media with antiviral protection of claim 1, wherein said antiviral compound or medicine includes a pyrophosphate analogue DNA polymerase inhibitor.

6. The in vitro fertilization media with antiviral protection of claim 1, wherein said in vitro fertilization media includes media for the isolation of oocytes, ovum and other cells of ovarian origin during the egg retrieval process.

7. The in vitro fertilization media with antiviral protection of claim 1, wherein said in vitro fertilization media includes media for the fertilization of ovum.

8. The in vitro fertilization media with antiviral protection of claim 7, wherein said media for the fertilization of ovum includes media adapted to fertilization of ovum by sperm-egg interaction.

9. The in vitro fertilization media with antiviral protection of claim 7, wherein said media for the fertilization of ovum includes media adapted to fertilization of ovum intracytoplasmic sperm injection.

10. The in vitro fertilization media with antiviral protection of claim 1, wherein said in vitro fertilization media includes media for the preparation and isolation of sperm cells used in the in vitro fertilization process.

11. The in vitro fertilization media with antiviral protection of claim 1, wherein said in vitro fertilization media includes media to sustain the viability of embryos of all stages and blastocysts from the time of fertilization until replacement in the uterus, embryo biopsy, embryo cryopreservation or any other ultimate disposition of the embryo.

12. The in vitro fertilization media with antiviral protection of claim 1, wherein said in vitro fertilization media includes media to sustain the viability of other cells which may be grown together in co-culture of human oocytes and embryos of all stages of development.

13. The in vitro fertilization media with antiviral protection of claim 1, wherein said in vitro fertilization media includes media used in the cryopreservation and thawing of human sperm cells, oocytes, ovum, embryos and blastocysts at all stages of development.

14. The in vitro fertilization media with antiviral protection of claim 1, wherein said in vitro fertilization media includes media used in the maintenance and transportation of biopsied embryo cells for further analysis or use.

15. The in vitro fertilization media with antiviral protection of claim 1, wherein said in vitro fertilization media includes media for the pre-conditioning of laboratory containers or catheters to be later used in the in vitro fertilization process.

16. The in vitro fertilization media with antiviral protection of claim 1, wherein said in vitro fertilization media includes media used for the transfer of sperm cells, oocytes or embryos into the uterus of a recipient at the time of an ART procedure such as intrauterine insemination or embryo transfer.
